(19) United States
(12) Patent Application Publication
(10) Pub. No.: US 2025/0012784 A1
(43) Pub. Date: Jan. 9, 2025

(54) METHODS OF DIAGNOSING AND TREATING OSTEOARTHRITIS

(71) Applicant: Academia Sinica, Taipei City (TW)

(72) Inventors: Chi-Huey WONG, Taipei City (TW); Ching-Chuan JIANG, New Taipei City (TW); Shang-Cheng HUNG, Taipei City (TW); Chang-Hsun HSIEH, New Taipei City (TW); Pi-Hui LIANG, Taipei City (TW); Ting-Jen CHENG, New Taipei City (TW); Hwai-I YANG, New Taipei City (TW)

(21) Appl. No.: 18/707,599

(22) PCT Filed: Nov. 11, 2022
(86) PCT No.: PCT/US2022/049671

§ 371 Date: May 5, 2024

Related U.S. Application Data
(60) Provisional application No. 63/278,475, filed on Nov. 11, 2021.

(51) Int. Cl. G01N 33/53; A61K 31/135; A61K 31/167; A61K 31/192; A61K 31/196; A61K 31/405; A61K 31/485; A61K 31/5415; A61K 31/573; A61K 31/616; A61K 31/635

(52) U.S. Cl. CPC G01N 33/5308; A61K 31/135; A61K 31/167; A61K 31/192; A61K 31/196; A61K 31/405; A61K 31/485; A61K 31/5415; A61K 31/573; A61K 31/616; A61K 31/635; G01N 2400/40; G01N 2800/105; G01N 2800/50

(57) ABSTRACT

Disclosed herein is a method for identifying and treating a subject having stages III or IV osteoarthritis (OA). The method includes steps of, determining the respective levels of chondroitin-4-sulfate (C4S) and chondroitin-6-sulfate (C6S) derived from a chondroitin sulfate (CS) chain of bikunin in a biological sample of the subject; calculating a risk score (RS) of the subject with the equation:

$$R S = \frac{1}{1 + {e}^{- f ( x )}} ,$$

in which f(x)=−9.3875+0.1356×(the age of the subject)+1.1493×(the level of C4S/the level of C6S) when the subject is a female, or f(x)=−10.3389+0.1356×(the age of the subject)+1.1493×(the level of C4S/the level of C6S) when the subject is a male; and treating the subject with an analgesic, a non-steroidal anti-inflammatory drug (NSAID), a corticosteroid or a combination thereof when the determined risk score is above 0.745.

### CROSS-REFERENCE TO RELATED APPLICATIONS

This application claims priority under 35 U.S.C. § 119(e) to U.S. Provisional application, U.S. Ser. No. 63/278,475, filed Nov. 11, 2021, which is incorporated by reference herein in its entirety.

### BACKGROUND OF THE INVENTION

### 1. Field of the Invention

The present disclosure in general relates to identification and treatment of osteoarthritis (OA). More particularly, to a method of identifying OA subjects based on their urinary chondroitin sulfate (CS) composition derived from a CS chain of bikunin in the subject's urine.

### 2. Description of Related Art

Osteoarthritis (OA) accounts for 33.6% of the population over 65 years old and is a leading cause of chronic disability. It is defined as “a disorder involving movable joints characterized by cell stress and extracellular matrix degradation initiated by micro- and macro-injury that activates maladaptive repair responses including pro-inflammatory pathways of innate immunity” by OARSI (Osteoarthritis Research Society International). Although the quality of the degenerative cartilage can be evaluated by conventional clinical examinations, radiographs and magnetic resonance images (MRI), most of these measurements are used at late stage of OA. As early OA is still manageable by daily life changes, such as weight loss and exercise. Thus, early diagnosis becomes an important factor to alleviate the progress of OA.

Many physiological and pathological processes such as inflammation, angiogenesis, metabolic are mediated by glycoaminoglycans (GAGs). GAG components of proteoglycans (PGs) are linear, sulfated polysaccharides containing disaccharide repeating sequence of an amino sugar (GlcNAc or GalNAc) and uronic acid (glucuronic acid (GlcA) or iduronic acid) to the core protein. There are five major classes of GAGs in human, such as keratan sulfate (KS), heparin (HP), heparan sulfate (HS), dermatan sulfate (DS), and chondroitin sulfate (CS). CS accounts for more than 80% of GAGs in articular cartilage, and is composed of disaccharide GlcAβ-1,3GalNAc. In the structure of CS, each GlcA can be 2-O-sulfated and GalNAc can be sulfated at C(O4) or C(O6) position. Chondroitin-4-sulfate (ΔDi4S; GlcAβ(1,3)-4-SO₃-GalNAc) and chondroitin-6-sulfate (ΔDi6S; GlcAβ)(1,3)-6-SO₃-GalNAc) are two of the most common disaccharide unit among the internal linkage of CS. There is evidence suggesting that CS in aging cartilage samples has increased level of sulfation at the C(O6) relative to the C(O4) of GalNAc. Changes in the distribution pattern of CS in cartilage may be important because the levels of ΔDi4S and ΔDi6S in joint fluids and cartilage have been used as markers of cartilage metabolism.

Bikunin is a serine protease inhibitor found in human amniotic fluid, plasma, and urine. Bikunin is post-translationally modified with a CS chain, O-linked to a serine residue (Ser₂₁₅ in AMBP) of the core protein.²¹ The urine bikunin-derived CS are terminated with GalNAc residues and comprised 27-39 monosaccharides with 4-7 sulfo-groups distributed along the polysaccharide chain. The linkage region tetrasaccharide is characterized to β-D-GlcA(1→3)-β-D-Gal4S(1→4)-β-D-Gal(1→4)-D-Xylol; and in the non-reducing end (NRE) trisaccharide, β-D-GalNAc-(1→3)-μ-D-GlcA(1→4)-β-D-GalNAc4S(1→). Recent studies have shown that the CS chain of bikunin played an important role in the physiological and pathological functions. It has been reported that sulfation of the urinary bikunin GAG are altered in inflammation and renal stone disease, thus, urinary bikunin GAG may serve as the disease markers. These characteristics can be assessed using anionic HPLC, which should facilitate diagnostic studies involving bikunin. In addition, bikunin was virtually undetectable in normal cartilage. By contrast, in the OA cartilage, superficial zone chondrocytes synthesized more bikunin that became detectable in the cartilage and synovial fluids. Since OA is a systemic joint degraded disease, loss of hemostasis of GAG would influence systemic GAG-bearing protein.

Thus, there exist in the related art a need of an improved method for identifying and treating a subject having or suspected of having OA. Continual efforts are needed to analyze systemic GAG-bearing protein.

In the present disclosure, we identify that CS composition in the urine bikunin may serve as a non-invasive, early diagnostic tool for identifying subjects with OA.

### SUMMARY OF THE INVENTION

In view of above, the present disclosure provides novel methods for identifying and treating a subject having OA.

Accordingly, the first aspect of the present disclosure is directed to a method for identifying and treating a subject having stages III or IV osteoarthritis (OA). The method comprises steps of,
(a) determining the respective levels of chondroitin-4-sulfate (C4S) and chondroitin-6-sulfate (C6S) derived from a chondroitin sulfate (CS) chain of bikunin in a biological sample of the subject;
(b) calculating a risk score (RS) of the subject with the equation of:

$$R S = \frac{1}{1 + {e}^{- f ( x )}} ,$$

in which
   f(x)=−9.3875+0.1356×(the age of the subject)+1.1493×(the level of C4S/the level of C6S) when the subject is a female, or
   f(x)=−10.3389+0.1356×(the age of the subject)+1.1493×(the level of C4S/the level of C6S) when the subject is a male; and
(c) treating the subject with an analgesic, a non-steroidal anti-inflammatory drug (NSAID), a corticosteroid or a combination thereof when the determined risk score of step (b) is above 0.745.

Examples of the biological sample suitable for use in the present method include, but are not limited to, blood, plasma, urine, and saliva. Preferably, the biological sample suitable for use in the present method is urine.

According to embodiments of the present disclosure, in the step (a), the respective levels of C4S and C6S in the biological sample are determined by high performance liquid chromatography (HPLC).

Examples of analgesic suitable for use in the present method include, but are not limited to, acetaminophen, codeine, tramadol, and the like.

Examples of NSAID suitable for use in the present method include, but are not limited to, aspirin, ibuprofen, naproxen, diclofenac, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, and mefenamic acid.

Example of corticosteroid suitable for use in the present method includes, but is not limited to, cortisol.

According to embodiments of the present disclosure, the subject suitable for use in the present method is a human.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1 are images of human OA cartilages analyzed by IHC staining, in which human cartilages specimens obtained from OA lesion (panels B and D) and far OA lesion (panels A and C) areas were stained for expression of chondroitin 4-sulfate (C4S) (panels A and B) and chondroitin 6-sulfate (C6S) (panels C and D) in accordance with one embodiment of the present disclosure; and

FIG. 2 depicts the receiver operating characteristic (ROC) analysis on the ratio of C4S and C6S in urine samples of OA patients among the OA group and the control group, in which (A) is the relationship between sensitivity and specificity, and (B) is the risk scores (RS) derived from the present risk equation among patients in the OA group and the control group.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### 1. Definitions

Unless otherwise indicated, the term “patient” or “subject” may be used interchangeably in the present disclosure, and refers to any animal. The animal can be a human subject, or a non-human subject. The subject may be a human, but can also be a mammal in need of veterinary treatment, e.g., domestic animals or game animals, farm animals, and laboratory animals (e.g., rats, mice, guinea pigs, primates, and the like). Usually the animal is a non-human mammal, such as a non-human primate. Non-human primates include chimpanzees, cynomologous monkeys, spider monkeys, and macaques, e.g., Rhesus or Pan. Domestic animals and game animals include cows, horses, pigs, sheep, deer, bison, buffalo, mink, felines (e.g., domestic cats, canines (e.g., dogs)), wolf and fox, avian species (e.g., chicken, turkey, and ostrich), and fish (e.g., trout, catfish, and salmon). According to embodiments of the present disclosure, the subject suitable for use in the present method is a human.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

Aspects of the present disclosure relate to the findings that the certain chondroitin sulfates (e.g., chondroitin 4-sulfate (C4S) and chondroitin 6-sulfate (C6S)) patterns, which is translated into a risk score (RS), may be used to determine whether a subject has osteoarthritis (OA) or not, particularly the late stages OA (e.g., stages III or IV OA).

Accordingly, the first aspect of the present disclosure is directed to a method for identifying and treating a subject having stages III or IV OA. Preferably, the identification part of the present method is conducted ex vivo in a biological sample derived from the subject; and the treatment part of the present method is conducted in vivo in the subject, if the subject is identified as an OA subject. The present method includes steps of,
(a) determining the respective levels of chondroitin-4-sulfate (C4S) and chondroitin-6-sulfate (C6S) derived from a chondroitin sulfate (CS) chain of bikunin in a urine sample of the subject;
(b) calculating a risk score (RS) of the subject with the equation of:

$$R S = \frac{1}{1 + {e}^{- f ( x )}} ,$$

in which
   f(x)=−9.3875+0.1356×(the age of the subject)+1.1493×(the level of C4S/the level of C6S) when the subject is a female, or
   f(x)=−10.3389+0.1356×(the age of the subject)+1.1493×(the level of C4S/the level of C6S) when the subject is a male; and
(c) treating the subject with an analgesic, a non-steroidal anti-inflammatory drug (NSAID), a corticosteroid or a combination thereof when the determined risk score of step (b) is above 0.745.

Examples of the biological sample suitable for use in the present method include, but are not limited to, blood, plasma, urine, and saliva. According to preferred embodiments of the present disclosure, the biological sample is urine.

According to embodiments of the present disclosure, in the step (a), the respective levels of C4S and C6S in a urine sample are determined by high performance liquid chromatography (HPLC).

Then, in the step (b), a risk score (RS) is calculated based on the ratio of the determined C4S and C6S levels, in which an empirically identified coefficient equation (C) is employed. The coefficient equation is as followed,

$$R S = \frac{1}{1 + {e}^{- f ( x )}} ,$$

in which f(x)=−9.3875+0.1356×(the age of the subject)+1.1493×(the level of C4S/the level of C6S) when the subject is a female, or f(x)=−10.3389+0.1356×(the age of the subject)+1.1493×(the level of C4S/the level of C6S) when the subject is a male.

According to preferred embodiments of the present disclosure, the risk equation is established based on logistic regression analysis on respective levels of C4S and C6S present in the urine among two groups of subjects, that is, the OA subjects and the control subjects. Then, the discrimination capability of the risk equation is evaluated by the ROC curve, which is a graphical plot for illustrating the diagnostic ability of a binary classifier system as its determination threshold varies. The ROC curve is created by plotting the true positive rate (i.e., “sensitivity”) against the false positive rate (i.e., “1-specificty”) at various threshold settings. In the present disclosure, the distribution of a marker (i.e., the C4S/C6S) among the two groups of subjects might overlap, thus, a cutoff value (i.e., a threshold value) is chosen to differentiate subjects in the overlapped population. Once a cutoff value is selected, then subjects independently having a score above the cutoff value is classified as “positive” with the disease (i.e., OA), while subjects independently having a score below the cutoff value is classified as “negative” with the disease. According to preferred embodiments of the present disclosure, the ROC curve has an area under the curve (AUC) of 86.6%, which translates to mean the risk equation has at least 86.6% probability of differentiating stages III or IV OA from non-OA controls. Using the value 0.745 as the cut-off point, 79.2% of the OA patients can be correctly diagnosed as OA while 78.3% of the controls can be diagnosed as not having OA.

Once the subject is identified to have OA, a suitable medicament may then be administered to the subject to ameliorate symptoms associated with OA (the step (c)). The medicament may be an analgesic, a non-steroidal anti-inflammatory drug (NSAID), a corticosteroid or a combination thereof.

Examples of analgesic suitable for use in the present method include, but are not limited to, acetaminophen, codeine, tramadol, and the like.

Examples of NSAID suitable for use in the present method include, but are not limited to, aspirin, ibuprofen, naproxen, diclofenac, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, and mefenamic acid.

Example of corticosteroid suitable for use in the present method includes, but is not limited to, cortisol.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

### Urine Samples and Cartilage Samples

Human urine samples were acquired from 209 end-stage OA patients, including 149 stage III to IV OA patients (age ranged between 53 to 87 years old) and 60 before stage III OA patients (age ranged between 23-86 years old). None of patients had chemotherapy or trauma to the knee. Urine specimens taken from stages III to IV of the OA patients who needed joint replacement were classified as “the OA group”, while the OA patients who did not exhibit any sign of knee joint disease before stage III were used as the control group. All specimens were obtained with informed consent. The OA patients included in the study also complied with the following criteria, (1) male or female, (2) skeletally matured between age 20 to 90, (3) without any allergies or major systemic or organ diseases. If subjects were pregnant, or had rheumatoid immunity or metabolic arthritis that appeared to have severe cartilage damage, they were excluded from this study.

Human cartilage samples were acquired from the knees of stage IV OA patients from total knee arthroplasty.

### Immunohistochemistry (IHC) Staining

Two full-thickness cartilage samples were harvested from medial femoral condyle (MFC) and lateral femoral condyle (LFC). The cartilage samples included the area from the surface of the articular cartilage to the subchondral bone. Cartilage samples were divided into two groups: lesion cartilage, harvested from the area adjacent to the most severely degraded cartilage (osteoarthritic area) in which the subchondral bone was exposed; and far OA cartilage, harvested from a non-arthritic area or early osteoarthritic area. The full-depth cartilage was removed from the cylindrical sample. The specimens were embedded within optimal cutting temperature (OCT) compound (OCT compound from Leica microsystems, Singapore) in cryomolds and snapped frozen in liquid nitrogen. Sections of 5 μm thickness were mounted onto glass slides. Then were predigested with chondroitinase ABC (0.2 U/mL in PBS; Sigma-Aldrich) at 37° C. for 30 minutes then at room temperature to allow the release of C4S and C6S epitope.

For immunostaining, the sections were fixed with ice-cold 100% methanol at −20° C. for 20 minutes and blocked with goat serum for 20 minutes at room temperature. The sections were then incubated with primary antibodies (#MAB2030 for chondroitin-4-sulfate (C4S) from Merck, #MAB2035 for chondroitin-6-sulfate (C6S) from Merck, Darmstadt, Germany) for one hour at room temperature, followed by incubation with the gout FITC-tag anti-Mouse IgG (H+L) secondary Antibody (1:2000; #62-6511, Invitrogen Corporation, Carlsbad, CA, USA) for 30 minutes at room temperature. Images of fluorescence were visualized using a Zeiss Axiovert 200M microscope and the intensity of signal was analysed by ImageJ a free, automated web-based image analysis application for scoring immune-stained slides where percentage was calculated based on (area of green image/area of Transmitted)×100%. The data were normalized to the data from the remote area.

Collection of Bikunin from Urine

Urine specimens were collected (appr. 200 mL) and centrifuged at 3000 g for 15 min at 4° C. and after the centrifugation the pellets were removed. The supernatants were dialyzed in buffer (150 mM NaCl, 20 mM Tris, pH 8.6) at 4° C. for 24 hours. Then, the dialysates were eluted in an anion chromatography with a DEAE column (running buffer, 150 mM NaCl, 20 mM Tris, pH 8.6; eluting buffer, 2 M NaCl, 20 mM Tris, pH 8.6, the Supporting Information S1) in a ÄKTA™ FPLC system (GE Healthcare Lifesciences) at 4° C. while monitoring conductivity and UV at λ₂₈₀. The isocratic flow rate for the column was 0.2 mL/min while 4 mL fractions were collected upon injection of the protein sample. 200 μL of eluents in each fraction were separated by electrophoresis in a 10% SDS-PAGE, transferred to PVDF membranes, blocked with 3% BSA in PBST. One of membrane was incubated with Coomassie blue at 4° C. for 1 hour. The other membranes were incubated with anti-bikunin antibody (1:2000 dilution with PBS, abcam, #ab4307) at 4° C. overnight. Membranes were washed with PBST and incubated with HRP-conjugated secondary antibodies including goat anti-rabbit (1.5:5000, Cell Signaling, #7074). The antigens were revealed by Ultrascence western substrate (Bio-Helix, Taiwan), and image detection was performed with ChemiDoc XRS system and Image Lab software (Bio-Rad Laboratories, Hercules, CA).

Preliminary results confirmed that fraction 14 of the urine samples exhibited significant level of bikunin (data not shown), and such fraction was used for subsequent determination of disaccharide C4S and C6S.

### Chondroitin Sulfate Disaccharide Assay

Fractions that showed expression of bikunin were pooled and lyophilized to dryness. After lyophilization, the solids were dissolved in water (100 μL). The mixture was added 10 μL proteinase K (5 mg/mL in PBS buffer, Sigma-Aldrich) and also were incubated at 37° C. over 12 h. Proteinase K was then inactivated by incubation at 90° C. for 10 min and then centrifuged at 12,000 g for 5 min at room temperature to remove the pellets. The post proteinase K treated samples were added into 100 μL of tris-acetate buffer and incubated with 2 μL (20 mU) of chondroitinase ABC (0.2 U/mL in PBS; Sigma-Aldrich) for 1 day at 37° C. Then, chondroitinase ABC was inactivated by incubation at 90° C. for 5 min. The samples were filtered through Amicon Ultra-0.5 (3 kDa) prior to HPLC analysis. HPLC analysis was performed on the Agilent 1260 Infinity II. The CS disaccharide standards and the 20 μL of digested samples were applied to a ZORBAX SAX analytical column (4.6 mm×250, 5 μm; Agilent). Eluent A was H₂O/TFA and eluent B was 1M NaCl solution (in H₂O/TFA). Both eluent A and B were acidified to pH 3.5 with trifluoroacetic acid. In the first 5 min, the column was eluted with 100% of eluent B, and the eluent B was increased from 5% to 25% by a linear gradient in the following 20 min. During 25-45 min, the eluent B was increased from 8% to 100% by a linear gradient. During 45-55 min, eluent A was increased from 0% to 100% by a linear gradient and maintained at 100% of eluent A during 55-75 min. The flow rate was maintained under 1.0 mL/min and the absorbance was measured at 232 nm.

### Statistical Analyses

Statistical analyses were performed through the logistic regression analysis and Receiver Operating Characteristic (ROC) curve analysis. A p-value of <0.05 was considered statistically significant.

### Example 1 Chondroitin Sulfate Expression in Oa Cartilage

In this example, the level of chondroitin sulfate (e.g., C4S and C6S) in OA cartilage was examined via immunohistochemical (IHC) staining as described in the section of “Materials and Methods”.

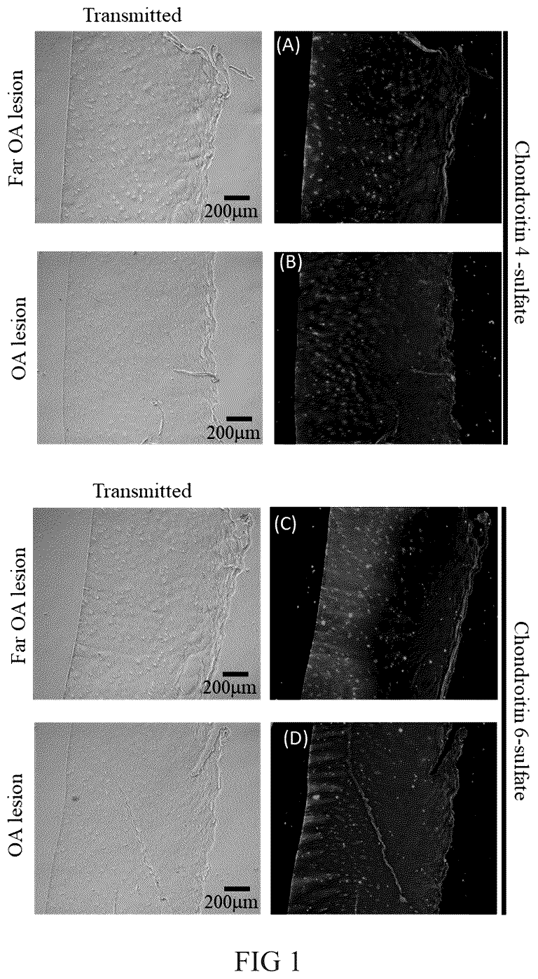

Note that even OA patients in the terminal phase of the disease still possessed some healthy cartilage, probably at the less weight bearing area of lateral femoral condyl (LFC). Accordingly, the lesion cartilages for used in this example were harvested from the area adjacent to the most severely degraded cartilage (i.e., OA lesion), while the far lesion cartilage (i.e., far OA lesion) was harvested from a non-arthritic area or early osteoarthritic area. Cartilages that had distinct differences in chondroitin-4-sulfate (C4S) between far OA lesion and OA lesion areas were selected for IHC image analysis. Results are depicted in FIG. 1.

The IHC results showed that the distribution of C4S and C6S in OA lesion area were more homogeneous than the remote area (i.e., far OA lesion). However, the immunofluorescent signal of C4S and C6S were generally stronger in the remote area than in the OA lesion area, especially in the lacuna. Additionally, optical density analysis via the software ImageJ showed that about 60% decreases of highly expressed C4S in the lesion cartilage, comparing to that in the remote area (i.e., far OA lesion). Furthermore, the lesion area had about 40% decreased highly expressed C6S level, comparing to that in the remote area.

### Example 2 Establishing Risk Factor Equation for Identifying Stages III or IV OA Patients

A total of 209 patients were enrolled in this study, in which 149 patients were classified as “OA group”, and 60 patients were classified as “the control group” in accordance with the criteria described in the “Materials and Methods” section.

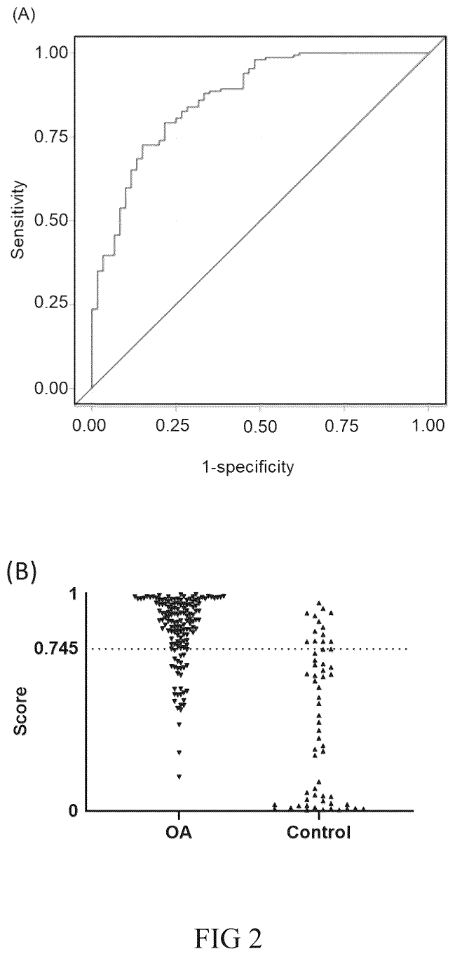

The patient information including gender, age, and di4/di6 ratio were subjected to multi-predictor logistic regression analysis so as to identify any relationship useful for identifying late stages OA between the analyzed factors. Results are summarized in FIG. 2 and Table 1.

TABLE 1
Multi-predictor logistic regression analysis
on OA patient information

| | Regression | Relative Risk of OA | p |
|---|---|---|---|
| Predictor | coefficient | (95% CI of Odds Ratio) | value |
| Intercept of the | −9.3875 | | |
| regression equation | | | |
| Gender | −0.9514 | 0.39 (0.17-0.88) | 0.0229 |
| (male vs female) | | | |
| Age, per year | 0.1356 | 1.15 (1.09-1.20) | <.0001 |
| C4S/C6S | 1.1493 | 3.16 (1.35-7036) | 0.0078 |

As summarized in Table 1, the proportion of women suffering from OA was higher than that of man; the relative risk of OA increased with age; and the ratio of Chondroitin 4-sulfate to chondroitin 6-sulfate increased with the risk of OA. Accordingly, equations for determining risk factor (RS) of OA were established as followed:

$$R S = \frac{1}{1 + {e}^{- f ( x )}} ,$$

in which,
f(x)=−9.3875+0.1356×(the age of the subject)+1.1493×(the level of C4S/the level of C6S) when the subject is a female, or
f(x)=−10.3389+0.1356×(the age of the subject)+1.1493×(the level of C4S/the level of C6S) when the subject is a male.

The ability of the above equation, which contains the ratio of C4S and C6S (i.e., C4S/C6S), served as an indicator for the diagnosis of OA was tested by receiver operating characteristic curve (ROC) analysis, and results are depicted in FIG. 2.

It was found that the value of area under the ROC curve (AUC) was 0.866, which has excellent discrimination (FIG. 2A). Further, if the risk factor of OA was set to 0.745, the sensitivity and specificity were 0.792 and 0.783, respectively (FIG. 2B).

Taken together, the results in this example confirmed that the level of CS, particularly the ratio of C4S/C6S, in urine may serve as an indicator for identifying subjects suffering from OA, particularly late stages OA (i.e., stages III or IV OA), so that suitable therapeutic or prophylactic medicament may be administered to such subjects.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A method for identifying and treating a subject having stages III or IV osteoarthritis (OA) comprising,
(a) determining the respective levels of chondroitin-4-sulfate (C4S) and chondroitin-6-sulfate (C6S) derived from a chondroitin sulfate (CS) chain of bikunin in a biological sample of the subject;
(b) calculating a risk score (RS) of the subject with the equation of:
$$R S = \frac{1}{1 + {e}^{- f ( x )}} ,$$
in which
f(x)=−9.3875+0.1356×(the age of the subject)+1.1493×(the level of C4S/the level of C6S) when the subject is a female, or
f(x)=−10.3389+0.1356×(the age of the subject)+1.1493×(the level of C4S/the level of C6S) when the subject is a male; and
(c) treating the subject with an analgesic, a non-steroidal anti-inflammatory drug (NSAID), a corticosteroid or a combination thereof when the determined risk score of step (b) is above 0.745.

2. The method of claim 1, wherein the biological sample is blood, plasma, urine, or saliva.

3. The method of claim 2, wherein the biological sample is urine.

4. The method of claim 1, wherein the respective levels of C4S and C6S in step (a) are determined by high performance liquid chromatography (HPLC).

5. The method of claim 1, wherein the analgesic is acetaminophen, codeine, or tramadol.

6. The method of claim 1, wherein the NSAID is selected from the group consisting of aspirin, ibuprofen, naproxen, diclofenac, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, and mefenamic acid.

7. The method of claim 1, wherein the corticosteroid is cortisol.

8. The method of claim 1, wherein the subject is a human.
